(19) United States
(12) Patent Application Publication
(10) Pub. No.: US 2023/0380706 A1
(43) Pub. Date: Nov. 30, 2023

(54) Pulse Detection Apparatus, System, and Method

(71) Applicant: Expulsus, LLC, Columbia, MD (US)

(72) Inventor: Patrick McCarville, Columbia, MD (US)

(21) Appl. No.: 18/143,786

(22) Filed: May 5, 2023

Related U.S. Application Data
(60) Provisional application No. 63/345,547, filed on May 25, 2022.

(51) Int. Cl. A61B 5/024; A61B 5/00

(52) U.S. Cl. CPC A61B 5/02416; A61B 5/0004; A61B 2562/0247; A61B 2562/06; A61B 5/681

(57) ABSTRACT

A pulse detection device including a substrate configured to be attached to a subject, the detection device including components for detecting a pulse and/or that couple monitoring of a heartrate, using photoplethysmography, with detection of a physical pulse force using a pressure sensor.

### CROSS-REFERENCE TO RELATED APPLICATION

This application claims the benefit of U.S. provisional application Ser. No. 63/345,547, filed May 25, 2022, currently pending, the disclosure of which are incorporated herein by reference.

### INTRODUCTION

Pulse detection is conventionally performed by an emergency (e.g. first responder) technician such as an EMS, and as such the accuracy of the detection can fluctuate based on the skill level and expertise of the technician detecting the pulse. Heartbeats create a pressure wave that distributed throughout the circulatory system and that can be sensed. The presence of a cardiac pulse in a patient is generally detected by sensing pulses from blood flow changes due to blood pumped from the patient's heart. In most cases, the technician uses their fingers to estimate a pulse force that is felt as the patient's heart beats. Fingers can, for example, be placed on a patient's neck area (e.g., carotid artery) and/or wrist area to sense their pulse. For example, one common manual technique used by an EMS technician is to place their index and middle fingers on the inner wrist of a patient, and counting the number of taps felt in a certain time period (e.g., 10 seconds), then multiplying that number by 6 to find out the heart-rate for one minute (e.g., beats per minute (bpm)).

Another technique for detecting blood pressure is by way of conventional blood pressure devices (e.g., sphygmomanometers that include a bulb, cuff, etc.) that can be used to determine the force or pressure exerted in the arteries by the blood as it is pumped around the body by the heart. Systolic pressure is pressure in the arteries during the period of the heart's contraction (e.g., the higher/top number), and diastolic pressure is the pressure in the arteries when the heart is relaxed, between heartbeats (e.g., the lower/bottom number). Blood pressure is conventionally/traditionally measured in millimeters of mercury (mmHg), which from a historical standpoint is a reference to how high the pressure in the arteries can raise a column of mercury in original sphygmomanometer designs. For example, so-called “normal” blood pressure should be less than 120/80 mmHg. But detection using a sphygmomanometer may be inaccurate due to improper sizing of the blood pressure cuff or placing the cuff over clothing.

Pulse pressure is the difference between the upper and lower numbers of blood pressure (e.g., 120−80=40 mmHg). Blood pressure and pulse pressure represent valuable health information, in particular in emergency situations and/or for continuous monitoring of a patient. For example, a high/wide pulse pressure is indicative of a wide difference between the top and bottom numbers. On the other hand, a low/narrow pulse pressure is indicative of a scenario of when pulse pressure is one-fourth or less of systolic pressure (the top number) (e.g., when a heart isn't pumping enough blood, such as in connection with heart failure and/or other heart valve diseases, or due to injury (loss of blood/internal bleeding)). For example, pulse pressure above the normal of 40 mmHg may be indicative of health (e.g., heart) problems. But pulse pressure derived from inaccurate blood pressure measurements can prevent accurate diagnosis.

Other factors may impact the accuracy of pulse detection, including patient-specific parameters (e.g., body weight), and situation-specific parameters (e.g., pulse detection is frequently conducted in emergency situations). For example, emergency situations present a challenging environment in which to accurately detect a patient's cardiac pulse. Such emergency situations typically introduce issues such as time constraints (since it is often the case that pulse detection takes place in a matter of seconds), and the overall stress of the emergency situation may contribute to rushed and therefore inaccurate initial pulse detection. At the extreme end, these pulse detection inaccuracies can lead to a scenario where a determination is made that a patient with a pulse does not have a pulse (which may trigger unnecessary steps such as defibrillation), or the opposite may occur (determining a pulse is present when it is in fact not present, and therefore foregoing potentially beneficial defibrillation). These inaccuracies can therefore have impacts far beyond mere misdiagnosis, and may play a life or death role.

Additionally, other known techniques such as arterial blood pressure detection are invasive as they require arterial catheters and the like to obtain pressure results. For example, while arterial lines provide the most accurate arterial blood pressure measurements, they are an invasive technique requiring insertion of a needle in an artery (e.g., such as radial, femoral, dorsalis pedis or brachial arteries). Moreover, this technique includes other requirements such needing to be connected to sterile (fluid-filled) system, which is connected to a device such as an electronic pressure transducer. Such invasive techniques are used, for example, when non-invasive techniques are not possible or when greater accuracy is required, or in situations where blood pressure must be maintained in very narrow range for a period of time, blood pressure is expected to fluctuate significantly, or continuous blood analysis is required. Non-invasive techniques may be in out-patient settings, and have advantages such as not producing wounds when compared to invasive techniques. However, they may produce less accurate results compared to invasive techniques. Arterial techniques also are not amenable to use in emergency situations such as when a quick pulse reading is needed (e.g., during ambulance transport, etc.).

In practice, the lack of a detectable cardiac pulse in a patient is a strong indicator of cardiac arrest (e.g., a life-threatening situation where the heart fails to provide enough blood flow to maintain life function). Defibrillation may be used to restart synchronized heart rhythm. But in the case where a patient lacks a detectable pulse but has detectable heart rhythm, techniques such as CPR may be used. Thus, it is important for a technician to be able to accurately detect a pulse so as to administer the appropriate treatment technique(s) (e.g., defibrillation therapy or CPR) that the situation calls for.

Additionally, continuous blood pressure monitoring (e.g., in non-emergency scenarios) is of great value. However, current blood pressure monitoring techniques also suffer from drawbacks, such as inaccurate detection, physical shifting of the monitor (e.g., on the body of a patient) such that blood pressure cannot be detected, and other problems.

In view of the above, there is a need for an apparatus, system and method that provides for quick and accurate determination of the presence/absence of a patient's pulse and/or heartbeat. Delays introduced by the inability to find and/or properly detect a patient's pulse and/or heartbeat can have a critical impact on the health of the patient.

### SUMMARY

This section provides a general summary of the disclosure, and is not a comprehensive disclosure of its full scope or all of its features.

The apparatus, systems and methods disclosed herein relate to pulse detection in a patient, and address the drawbacks of the art by using a plurality of (e.g., pressure) sensors for pulse detection purposes and/or by coupling the monitoring of a heartrate using photoplethysmography with the detection of a physical pulse force using a pressure sensor, in a unitary pulse detection device. Blood flow can be detected via use of the photoplethysmography techniques and a pulse force can be detected via use of the pressure sensing techniques.

In one embodiment, a plurality of pressure sensors (e.g., configured in an array) are implemented as pressure sensing components for blood pressure detection. A device configured with such a sensor arrangement has utility in applications including, but not limited to, continuous blood pressure monitoring, or in emergency scenarios such as described above. Such continuous blood pressure monitoring may be conducted in a hospital setting, but is not limited to such, and can be conducted in other settings such as the home or elsewhere, even in non-stationary applications. The sensors may be arranged on a common substrate that may be configured to be applied to the skin of a subject (e.g., a patient) for purposes of detecting a pulse of the subject.

In another embodiment, photoplethysmography techniques are implemented using light-emitting components (e.g., LEDs) as a light source and photodetectors (e.g., photodiodes) as (light) sensors, taking advantage of the fact that blood absorbs certain colors of light, and that light absorption changes during and between heartbeats. Pressure sensing components may be used in combination with the light emitting/detecting components, and serve as a substitute for a manual pulse check. These components may be arranged on a common substrate that may be configured to be applied to the skin of a subject (e.g., a patient) for purposes of detecting a pulse, etc. of the subject. Additional embodiments are provided below, but are not limiting as other embodiments are envisioned and encompassed within the scope of the present disclosure.

Another embodiment includes a detection apparatus comprising: a substrate; a processor; and a plurality of sensors, the processor and sensors being associated with the substrate; wherein the sensors are configured to sense a parameter of a subject to which the substrate is associated with, the processor is configured to receive and process the output of the sensors and to output data representing a status of the subject, such that the status of the subject can be indicated to a user of the detection apparatus.

Another embodiment includes a detection system comprising: a detection module; and a remote terminal, wherein the detection module comprises: a substrate; a processor; a transmitter; and a plurality of sensors, the processor, transmitter, and sensors being associated with the substrate, wherein the remote terminal comprises: a processor; a receiver; and an indicator, wherein the sensors are configured to sense a parameter of a subject to which the substrate is associated with, the detection module processor is configured to receive and process the output of the sensors and output, via the transmitter, data representing a status of the subject, and the remote terminal, via the receiver, is configured to receive the transmitted data representing the status of the subject, process, via the processor of the remote terminal, the received data representing the status of the subject, and indicate, via the indicator, a user-perceivable representation of the processed data, such that the indicator indicates the status of the subject to a user of the detection system.

Another embodiment includes a method for detection, the method comprising: attaching a detector to a subject; acquiring a parameter of the subject via sensors of the detector; evaluating the parameter to determine a status of the subject; and indicating the status of the subject.

Another embodiment includes a detection apparatus comprising: a substrate; a processor; and at least one sensor, the processor and the at least one sensor being associated with the substrate; wherein the at least one sensor is configured to sense a parameter of a subject to which the substrate is associated with, the processor is configured to receive and process the output of the at least one sensor and output to the indicator data representing a status of the subject, for indication of the status of the subject to a user of the detection apparatus.

Another embodiment includes a detection system comprising: a detection module; and a remote terminal, wherein the detection module comprises: a substrate; a processor; a transmitter; and at least one sensor, the processor, transmitter, and at least one sensor being associated with the substrate, wherein the remote terminal comprises: a processor; a receiver; and an indicator, wherein the at least one sensor is configured to sense a parameter of a subject to which the substrate is associated with, the detection module processor is configured to receive and process the output of the at least one sensor and output, via the transmitter, data representing a status of the subject, and the remote terminal, via the receiver, is configured to receive the transmitted data representing the status of the subject, process, via the processor of the remote terminal, the received data representing the status of the subject, and indicate, via the indicator, a user-perceivable representation of the processed data, such that the indicator indicates the status of the subject to a user of the detection system.

Another embodiment includes a method for detection, the method comprising: attaching a detector to a subject; acquiring a parameter of the subject via at least one sensor of the detector; evaluating the parameter to determine a status of the subject; and indicating the status of the subject.

Another embodiment includes a detection apparatus comprising: a substrate; a processor, the processor being external to the substrate; and at least one sensor, the at least one sensor being associated with the substrate; wherein the at least one sensor is configured to sense a parameter of a subject to which the substrate is associated with, the processor is in operative communication with the at least one sensor and is configured to receive and process the output of the at least one sensor and to output data representing a status of the subject, such that a user of the detection apparatus can determine the status of the subject via the output data representing the status of the subject.

Another embodiment includes a detection apparatus comprising: a sensor configured to sense a parameter of a subject; and circuit components configured to receive, store, and/or analyze data acquired by the sensor for determination of (i) the parameter and/or (ii) a status of the subject.

Another embodiment includes a detection system comprising: a detection device; a terminal separate from but in operative communication with the detection device, wherein the detection device is configured to be used in association with a subject, and comprises (i) a sensor configured to sense a parameter and/or a status of the subject, and (ii) circuit components configured to receive, store, and/or analyze data acquired by the sensor.

Another embodiment includes a detection method comprising: sensing, via a sensor, a parameter of a subject; and receiving, storing, and/or analyzing, via circuit components, data acquired by the sensor for determination of the parameter and/or a status of the subject.

Another embodiment includes a computer-implemented method for training a model for use in pulse detection applications, the method comprising: performing, by a processor, the steps of: receiving training data, wherein the training data comprises known pulse detection parameters for a plurality of known pulse events; generating a plurality of features from the training data; processing the features and determining pulse events and/or pulse parameters from the processed features; and creating a model for predicting pulse events and/or pulse parameters based on the determined pulse events and/or pulse parameters.

Additional embodiments include: (i) a method comprising any of the steps described herein; (ii) a system configured to perform any of the steps described herein; (iii) an apparatus configured to perform any of the steps described herein; and (iv) a computer program product embodied on a non-transitory computer readable storage medium comprising executable instructions, which when executed, performs any of the steps described herein.

In the above-described embodiments: the sensors may comprise optical sensors, LEDs, photosensors, and pressure sensors; the sensors may comprise only pressure sensors; the sensors may be integral with the substrate; the parameter of the subject may be a pulse parameter of the subject; and the status of the subject may be a physiological status of the subject. The sensor(s) according to any of the above embodiments may comprise: (i) one of an LED and photosensor pair, and a pressure sensor; (ii) a pressure sensor; and (iii) the sensor being integral with the substrate. The parameter of the subject of the above-described embodiments may comprise the parameter of the subject being a pulse parameter of the subject. The status of the subject of the above-described embodiments may be a physiological status of the subject. The substrate of the above-described embodiments may be configured to be attached to the subject.

These are merely some of the innumerable aspects of the present invention and should not be deemed an all-inclusive listing of the innumerable aspects associated with the present invention. These and other aspects will become apparent to those skilled in the art in light of the following disclosure and accompanying drawings. The description and specific examples in this summary are intended for purposes of illustration only and are not intended to limit the scope of the present disclosure.

### BRIEF DESCRIPTION OF THE DRAWINGS

The accompanying drawings, which are incorporated in and form a part of the specification, illustrate the embodiments of the present disclosure and together with the description, serve to explain the principles of the disclosure.

FIG. 1A illustrates a bottom plan view of a pulse detection instrument according to one embodiment.

FIG. 1B illustrates a side view of the pulse detection instrument of FIG. 1A.

FIG. 1C illustrates an embodiment of the sensor of the pulse detection instrument of FIGS. 1A and 1B.

FIG. 1D illustrates additional aspects of the sensor embodiment of FIG. 1C.

FIG. 1E illustrates an alternative embodiment of the pulse detection instrument using a strap.

FIG. 2 illustrates a bottom-centric perspective view of another embodiment of a pulse detection instrument.

FIG. 3A illustrates an embodiment of the pulse detection instrument including a visual indicator.

FIG. 3B illustrates an embodiment of the pulse detection instrument including an audible indicator.

FIG. 3C illustrates an embodiment of the pulse detection instrument including a tactile/haptic indicator.

FIG. 4 illustrates a circuit schematic according to an embodiment of the pulse detection instrument.

FIG. 5 illustrates a control program process flow according to an embodiment of the pulse detection instrument.

FIG. 6 illustrates an alternative embodiment of the pulse detection device utilizing wireless data communication.

FIG. 7 illustrates a circuit schematic according to a wireless communication embodiment of the pulse detection instrument.

FIG. 8 illustrates a control program process flow according to a wireless transmission embodiment of the pulse detection instrument.

FIG. 9A illustrates an additional embodiment of a pulse detection device that utilizes a pulse detection model.

FIG. 9B illustrates a relationship between computer components of the pulse detection device according to FIG. 9A.

FIG. 9C illustrates an example process flow for training a model according to FIGS. 9A and 9B for determining pulse information based on training data that comprises known pulse information.

### DETAILED DESCRIPTION

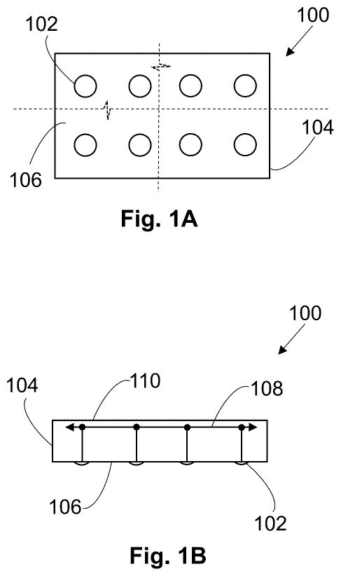

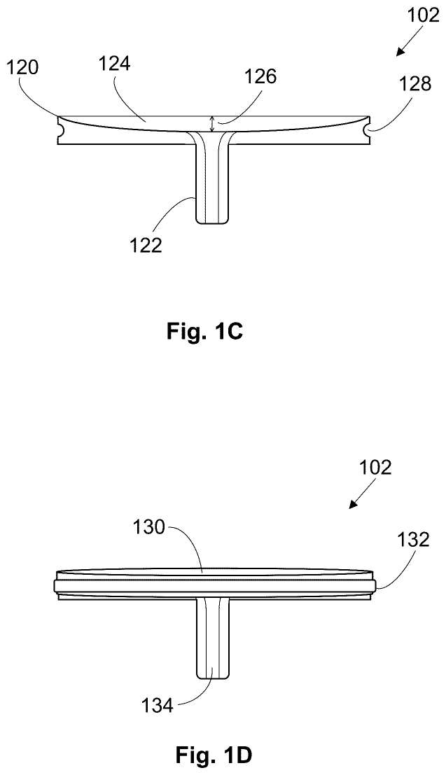

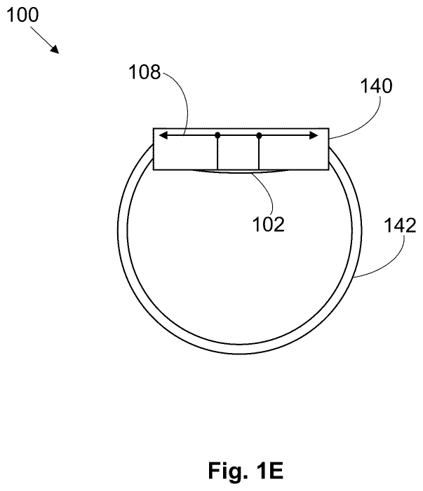

FIG. 1A illustrates a bottom view of one embodiment of a pulse detection instrument/device 100. The device 100 may comprise an array of components 102, with each individual component 102 being spaced from adjacent components 102 in a predetermined arrangement. As described below in more detail, these components 102 may comprise a plurality of LEDs, photodetectors, and/or pressure sensors. In certain embodiments, the components 102 may all be of the same type, e.g., all comprising pressure sensors, whereas in other embodiments the components 102 may comprise a mixture of sensing components (e.g., pressure sensors and light emitting/detecting components). The device 100 comprises a substrate 104 having length, width and thickness dimensions that are suitable for being applied to pulse detection portions of the human body, such as the neck area and/or the wrist area. For example, in one embodiment, the substrate 104 may be sized to be 4×6 inches with a thickness similar to a gauze pad or other bandage for application to the neck area. In another embodiment, the substrate may be of a smaller size, so as to be more amenable to placement on the wrist area. The dashed broken lines in FIG. 1A illustrate that the length, width, thickness, etc. and the amount of components may be variable and set to the needs of the particular application. In use, bottom surface 106 of the device 100 is applied to the skin of a subject (e.g., patient). The bottom surface 106 may comprise adhesive (e.g., a strip of adhesive around the perimeter of bottom surface 106, not shown) so as to aid in attachment and firm hold of the device 100 onto the subject's intended body portion for pulse detection purposes. The device may be attached to a subject in other ways, such as via straps or other securing mechanisms, or even by way of the user/technician simply holding the pad on the subject with their hand so as to be able to acquire a reading (e.g., pressing the pad against the desired location on the subject's body).

Referring to FIG. 1B, the substrate 104 may comprise a single layer of material or be comprised of multiple layers. This may include use of various textile and/or plastic materials capable of flexing or otherwise pliable enough to be able to conform to portions of a human body, similar to conventional bandages (e.g., gauze pads) that have an adhesive portion that adheres to the skin and a pad portion that covers a particular portion. However, other materials and combinations of materials may be used, for example gauze-like or other threaded/weaved textile-type materials may be used. If the substrate comprises multiple layers, the layers may be of the same material or of different materials. In any case, the components 102 may be arranged to protrude outwardly from a bottom surface 106 of the substrate 104 so as to be able to sufficiently interact with the skin of the subject (e.g., patient) to which the device 100 is applied. Such protrusion may be very slight, such that the faces of the components are effectively flush with the bottom surface 106, but still capable of making sufficient contact with the skin of the subject to which device 100 is applied to. Various electrical circuitry 108, described in more detail below, may run through/between any individual layer or combination of layers of device 100, similar, for example, to traces and through-holes present in circuit board fabrication and design. Also shown is top surface 110 of the device 100.

Further regarding components 102 and circuitry 108, the components 102 may comprise any mix of optical sensors, (for example LEDs (e.g., green LEDs) and photodetectors (e.g., photodiodes)), and/or pressure sensors, in combination or individually by type. These are preferred types of sensors, but other sensors including accelerometers, position sensors, and the like are also within the scope of usable sensors, for example for sensing other events (e.g., movement) beyond any core heart/pulse events. For example, the components 102 may comprise only pressure sensors, but the pressure sensors may be the same or of different types having different sensing capabilities. Or the pressure sensors can be grouped with optical sensors (e.g., light emitting/detecting components) in a co-operative manner. Similarly, amongst the optical components, any combination of LEDs and/or photodiodes can be used, including like components (e.g., all with the same functionality), or, for example a mix-and-match of light components (e.g., LEDs/photodiodes) of various different (e.g., detecting) capabilities (e.g., LEDs of various wavelengths, photodiodes of various detection, etc.). While it is preferable to use a plurality of components 102, use of only one component 102 (e.g., one sensor) is acceptable if the desired monitoring/sensing can be achieved. This group of components 102 may also be generally referred to as sensors, and the sensors may be referred to as optical sensors, pressure sensors, motion sensors, etc. The LEDs and photodiodes may be used to achieve photoplethysmography (PPG) functionality of device 100, whereas the pressure sensors may be used to achieve the pressure sensing functionality of device 100. Circuitry 108 may comprise a printed circuit board. For example, the circuit board may be located within (e.g., a layer of) the substrate 104, or between layers of the substrate 104. The printed circuit board of circuitry 108 may comprise conductive wiring, traces, contacts and/or other through-holes and landings in, on, and/or through the substrate 104 for signal transmission purposes. In place of or in addition to a rigid circuit board, circuitry 108 may comprise flex-wiring capable of being flexed along with the overall flexing capabilities of the substrate 104 of the device 100. The circuitry 108 may comprise, for example, only wires/conductors for signal receipt/transmission, e.g., of the (e.g., electrical) output(s) from each component 102, e.g., no formal circuit board structure is required. Each component output may be coupled to a bus (e.g., data bus) for transmission of the data representing the blood flow and/or pressure, etc. as sensed/detected by the component(s) 102. The bus may be a bus (e.g., trace) of a circuit board or other common conductor (e.g., not of a circuit board) capable of performing as a data bus. The circuitry 108 therefore is not limited in the form it takes, but should in all cases provide for the necessary signal transmission. The circuitry 108 therefore is in operative communication with the components 102 so that the physical parameters sensed by the components 102 can be received and processed by the circuitry 108. Although not shown in detail in FIG. 1B, the circuitry 108 may also comprise electronic components such as microprocessors and other IC's (e.g., for signal conditioning/processing, etc.), as well as other components such as an antenna (e.g., for wireless data transmission) and a battery (e.g., thin-profile battery) for powering of the device 100. In an embodiment where no circuit board structure is present, such components may be strategically placed within the body of the pad/substrate. For example, the pad/substrate may have dedicated portions for receipt of electronic components. These are mere examples and not limiting. For example, in one embodiment the circuitry 108 includes a rigid printed circuit board located within the substrate 104 (e.g., pad), where sensors 102, connected to the circuit board 108, extend from the substrate so as to protrude outward from the bottom surface of substrate 104 in order to be able to make contact with the body section of the subject to which the substrate is applied to. In another embodiment, because a rigid circuit board may impact the ability of the pad to flex in a manner necessary for matching (e.g. conforming to) the shape/contour of the body portion to which the pad is attached, the circuitry 108 may be a flex-circuit capable of flexing with the pad so that any impact of the physical circuitry on obtaining the desired contact between the sensors and (the skin of) the subject is minimized. For example, this embodiment may comprise a flex-circuit located near the surface of the substrate that interfaces with the subject, wherein surface-mount pressure sensors may be connected with the flex-circuit. Such an embodiment would be preferable for purposes of having a device that maintains flexibility and has a small-size. In yet another embodiment, a mix of rigid and/or flexible circuit boards/wiring may be used, and/or external computing hardware and/or other equipment may be connected to the circuitry located within the pad. In yet another embodiment, the components 102 (not on/in a circuit board, flex-circuit, etc.) may be received in a dedicated portion of the pad so as to face outward to be placed in contact with a patient, and wires, connected to an end of the components 102, may run from the ends of the components 102 to other portions of the pad/substrate. The connection between internal electronic components of the pad and any external electronic components may be done by way of physical connectors, leads, wires, tubing, and the like, or via wireless transmission (for example, the sensor data may be acquired by a dedicated IC in the pad, and converted to a (digitized) data format capable of wireless transmission, where the pad may have a simple antenna therein which is in operative communication with the IC, for transmission of the converted sensor data to external processing equipment via the antenna, for further processing of the sensor data). For example, the pad may only have the sensors (and associated cabling/wiring/tubing) located therein, and any electronics associated with the sensors may be located external to the pad but still in (e.g., mechanical and/or electrical) communication with the sensors. For example, in one mixed embodiment, a (rigid) circuit board may be configured in/on/at one layer (e.g., the upper surface) of the pad, whereas the sensors may be configured in/on/at another layer (e.g., the bottom surface) of the pad so as to make contact with the subject. Because the circuit board is located at the upper portion of the pad, the bottom portion of the pad closest to the subject's body will still be able to conform to the shape/contour of the body portion to which the pad is applied, e.g., assisted by the conformance/pliability of the pad and any adhesive thereof. The outputs from the component(s) 102 may be input into a processor (e.g., microcontroller) for processing of the outputs and to make determinations as to the strength/weakness of the detected pulse (e.g., the quality of the detected pulse), such processor being part of circuitry 108, for example. The output from each sensor may be conditioned (e.g., amplified) and fed to processing circuitry that processes the detection information of any sensor, for transmission of such detection data to a nearby (e.g., wireless) device such as a mobile computer terminal or other computing device. Such mobile device may have software stored thereon capable of displaying the received sensor data via a Graphical User Interface (GUI) of the mobile device, so that a technician using the device 100 and the mobile device can read and react to the sensed data accordingly in order to make a determination as to the patient's status. In the context of an embodiment directed to continuous blood pressure monitoring, the components 102 may only be pressure sensors, and may be used in non-emergency settings to continually monitor blood pressure, but still provide the necessary data read-outs to provide for accurate, continuous monitoring that is easy to perceive (e.g., see/hear) by a user. In the context of continuous monitoring, the user may be a technician in a hospital (e.g., nurse, etc.) in charge of monitoring a patient, or, in a home setting, the user may be the actual person that the device is attached to. In other words, an at-home user may be the one monitoring the detection results output from the device.

In a preferred embodiment shown in FIGS. 1C and 1D, the sensor 102 may comprise a pressure sensing member that includes a head 120 and a stem 122. The head 120 may comprise a concave (e.g., bowl) portion 124 of a certain depth 126. For example, the depth may be 0.5 mm deep, or 2.0 mm deep, although these example depths are not limiting (and smaller sizes are envisioned). The head 120 also includes cutout portion 128, which allows for the securing of a diaphragm member (discussed below) to the head. This portion 128 is effectively an indent/groove around the circumference of the head (although the head is not limited to a circular shape). This embodiment of the sensor 102 (e.g., head 120, stem 122, etc.) may be made of a suitable plastic material, but the type of material is not limited to this.

FIG. 1D shows the pressure sensor 102 of FIG. 1C, but with a diaphragm (or membrane) member 130 attached. The diaphragm member 130 may be secured to the head 120 via use of an elastic band 132 or other securing mechanism, via the cutout 128. For example, a diaphragm 130 may be placed over head 120 to an extent that covers the bowl 124, and with edges of the diaphragm being secured to the head 120 when the elastic band 132 is seated in the cutout 128. This sealing of the diaphragm over the bowl creates a fixed volume of air, which can be utilized as part of the pressure sensing mechanism for this embodiment. For example, the diaphragm is tightly stretched and sealed across the open face of the bowl, and can serve to sense pressure changes by way of deflection/movement of the diaphragm 130. The diaphragm 130 may comprise a material such as latex, but is not limited to such. Any material capable of use in flexing under pressure is capable of being used. Although the above embodiment secures the membrane to the head via a securing member such as an elastic band, other manners of securing the membrane to the head are envisioned, and the particular securing configuration of FIGS. 1C and 1D is therefore not limiting. For example, a membrane may be secured to a top surface of the head via adhesive or other sealing technique. In such a configuration, there may be no need for cutout 128 as the membrane will be sufficiently secured to a top (or side) surface of the head.

In operation, in the embodiment of FIGS. 1C and 1D, the head may be pressurized, and the membrane is pressed against a patient's neck or other skin surface for purposes of pulse detection. Due to the pressurization, the membrane will move in accordance with the pulse, and such movement can be detected and translated via corresponding electronic sensing units to indicate the presence (and even strength) of a pulse. For example, the physical deflection of the membrane may be sensed and the data representing such deflection may be transmitted to and used in association with any circuitry 108, such that the circuitry can convert the sensed physical parameters to digital information for downstream processing. For example, the pressure differentials resulting from the membrane being pressed against the patient's neck can be translated as described herein to provide a user (e.g., technician) of the device with indication of the presence (or absence) of a pulse, in the manners described herein (e.g., where the final indication of the pulse status may be communicated to the technician by way of visual indicator, audible alert, etc.). The accuracy of the pulse detection in this embodiment is dependent upon the membrane properties, and other aspects such as the bowl depth. The accuracy is also dependent on aspects such as the force with which the membrane is pressed against a patient's skin. As shown in FIG. 1D, the stem 122 may have a hollowed out interior 134 to allow for pressure/air changes due to deflection of the membrane to be transmitted downstream to another device (e.g., a component of 108) capable of parsing the physical pressure and translating this to usable electronic information for further processing. The end of the stem 122 may have a nipple or other structural feature on the outer surface thereof (not shown) to allow for secure interfacing of the stem with another component/device (e.g., via tubing, etc.) as needed. The head/membrane combination may be pressurized to assist in detection of deflections and corresponding force measurements, and such pressurization may be from an external source (e.g., air tank or other gas source). A nipple for external pressurization (not shown) may be present on the head, for example. Alternatively, the head can be pre-pressurized (e.g., sealed) to a desired level, and configured to maintain pressurization up until and/or during use for accurate detection, e.g., for a set number of uses (e.g., until the pre-pressurization is no longer at a sufficient level). The substrate/pad may therefore be configured to have tubes running therethrough and/or into or out of the substrate/pad, for use in connection with such pressurized aspects. The tubes provide for gas (e.g., air) flow as needed in order to be able to accurately determine pulse force/changes via deflection of the membrane, for example. For example, tubing may be run through the layers of pad/substrate 104 in a similar manner as circuitry 108 is depicted in FIG. 1B.

FIG. 1E illustrates an embodiment of device 100 where a single sensor 102, such as described above in connection with FIGS. 1C and 1D, may be positioned in/at a bottom face of a housing 140 of the device. For example, the bottom of the housing 140 may contain an aperture (not shown) that is sized to fit the head 120 of the sensor 102, so that the diaphragm 130 is exposed from the housing for contact with a skin surface. Associated circuitry 108 (or tubing, as described herein) may be within housing 140, and may comprise components for facilitating transmission and/or other processing of the detected pressure from the diaphragm. A strap 142 may be connected to housing 140 to allow the device 100 to be secured to a patient. The strap 142 may be adjustable so that a tighter/looser fit can be obtained as needed to arrive at a desired contact/pressure between the face of the sensor 102 and the patient's skin. The strap 142 may comprise a buckle or other adjustment mechanism (not shown) to allow for adjustment of the strap (e.g., to adjust tightness about a body part to improve accuracy of pressure readings). For example, a tighter strap may create a better contact between the sensing portion of the pressure sensor and the skin, resulting in more accurate readings. The circuitry 108 may be configured to detect a tightness of the strap 142 to give a confidence rating as to if the strap is tight enough for the sensor to accurately sense and return accurate pressure results. For example, a position sensor inside of housing 140 may be configured to detect the position of strap 142 and determine a tightness based on the detected position of the strap, and correlate the tightness/position of the strap to a degree of anticipated accuracy of pressure detection via sensor 102. The strap 142 may generally be sized to fit around a patient's wrist or neck. In the scenario where the strap is configured for use around a neck, the strap may be detachable and/or able to be separated (e.g., at a junction point of the strap) so that the strap can be detached to allow for placement of the strap around the neck, and then re-attached to secure the strap around the neck. For example, a buckle or other fastening mechanism may be used at a breakpoint near the middle of the strap, or any other beneficial/useful location. The embodiment in FIG. 1E differs from the above-discussed adhesive pad embodiment(s), in that the sensor(s) 102 may be within a housing, and the housing may be secured to the patient by way of the adjustable strap that can be tightened so as to arrive at an optimal sensor-to-skin pressure/contact threshold. For example, in the adhesive pad embodiment, the adhesive sticking to the skin may provide/maintain the necessary contact force between the sensor(s) and the skin. In the embodiment of FIG. 1E, the strap may provide such necessary contact force between the sensor(s) and the skin. For example, the housing 140/strap 142 combination may be configured similar to a wearable device such as a (smart) watch, especially in the embodiment where the device is configured for wrist detection. In the case of neck detection, the housing/strap and/or sensor 102 may be sized to be larger than the wrist embodiment. For example, a sensor face in the wrist embodiment may be smaller a sensor face in the neck embodiment since the area to which the wrist sensor is to be applied is smaller than the neck area. In place of or in addition to a buckle for strap adjustment, the device may be configured with other technical features capable of increasing/decreasing a tightness of the strap around a patient's body part (e.g., neck, wrist). For example, similar to conventional blood pressure units that use a pump mechanism, there may be a pump/tube mechanism for setting/adjusting the tightness of the strap (e.g., the strap may have a bladder therein that can be filled with air from a pump bulb to change the tightness around the body part). Other tightening features are envisioned, such automatic tightness adjustments (e.g., as opposed to manual adjustments). For example, instead of a user manually tightening the strap using a buckle, the device may have a teeth/gear assembly (not shown) in conjunction with a motive force (e.g., motor) that can auto-tighten the strap. For example, a motor may cause movement that pulls in or releases the strap via translation of a gear/teeth assembly to adjust tightness. The tightness of the strap may be monitored by position sensors capable of detecting the strap position and correlating the detected position to tightness parameters. The device may be configured such that when the detected tightness reaches a suitable threshold, an indication is triggered, wherein such indication in the manual adjustment context alerts a user to stop manual adjustment of the strap, and in the automatic adjustment context may transmit a signal to a corresponding controller/IC of the device to stop automatic adjustment of the strap. In any of the above-described strap embodiments, there may be (e.g., pressure) valves or other pressure-related components for use in arriving at the desired strap adjustment and pressure properties. These are mere examples of strap tightening implementations, and other variants are envisioned. While FIG. 1E only depicts one sensor, plural sensors may be used.

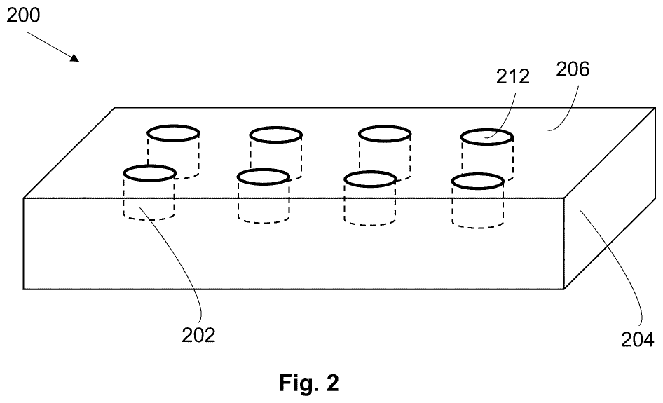

FIG. 2 illustrates a perspective view of an embodiment of a pulse detection device 200, similar to device 100, comprising an array of components 202 (like reference characters (e.g., 100/200, 102/202, etc.) may be used in a common manner across different figures herein). As discussed above, any suitable number of sensors 202 in any mix/match configuration may be used. As shown, the sensors 202 comprise a face 212. In the case where the sensor 202 is an LED or photodiode, the face 212 may be partially translucent to permit passage of the necessary light (e.g., for emitting/receiving) for photoplethysmography purposes, which may be implemented using green LEDs as a light source and photodiodes as light sensors, taking advantage of the fact that blood absorbs green light, and that light absorption changes during and between heartbeats. Thus, the LEDs and photodiodes may be configured in such a manner so as to be able to achieve the necessary interactions with the subject's skin for purposes of blood flow (e.g., heartrate) detection. However, LEDs have certain drawbacks such as being less accurate when used with darker skin tones (e.g., skin with more melanin blocks green light, making it harder to get an accurate reading). Thus, while use of LEDs is described and envisioned herein, other embodiments may forgo LEDs in favor of only using pressure sensors for detection purposes, thereby avoiding such LED shortcomings. In the case that sensor 202 is a pressure sensor, the face 212 effectively serves as a substitute for a manual (e.g., finger) pulse check (e.g., for pulse force). Thus, face 212 of the pressure sensor should be configured in such a manner so as to be able to have direct contact with a subject's skin to realize the pulse-check function, or any other configuration best-suited for pulse detection. The faces 212 may be effectively flush with bottom surface 206. Substrate 204 as shown in FIG. 2 is only a partial portion of the overall substrate of the device 200, where device 200 may be approximately 4×6 inches in size, with adhesive (not shown) located around the perimeter of surface 206, and with the array of pressure sensors and/or LED/photodiode components distributed about the surface that is attached to the subject's skin in such a manner so as to reduce the need to have precise placement on the subject's body, since a signal for both heartrate and pulse force can come from anywhere within the substrate coverage area. However, the pad of device 200 (or any other embodiments) is not limited to this shape/size and can comprise other shapes/sizes suitable for application on a human body (e.g., neck/wrist area). For example, device 200 could be a 3 inch diameter circle, 5 inch diameter circle, etc. While a plurality of sensors 202 is shown, use of one sensor is also envisioned.

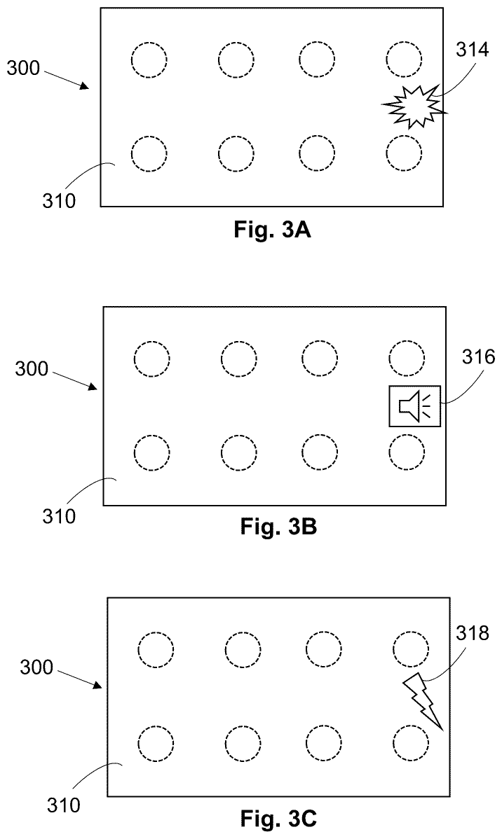

FIGS. 3A, 3B and 3C illustrate another embodiment where device 300 may comprise an indicator configured to alert the technician that a sufficient heartrate and/or pulse force was detected. The indicator may be any one or combination of a visual, audible, and/or tactile/haptic indicator, and may be arranged relative to top surface 310 so that the technician can more readily see, hear, etc. the indicator. For example, the indicator serves as a quick reference for the technician to see/hear/feel in terms of being able to quickly ascertain the presence/absence of heartrate and/or pulse force. FIG. 3A illustrates a visual indicator 314, such as an LED that may emit different colors relative to the presence/absence of the various quantities (e.g., heart function, pulse function) being detected. The visual indicator 314 may alternatively be a screen/display or other component that provides for displaying of visual information. FIG. 3B illustrates an audio indicator 316, such as a speaker, buzzer or other sound generating device that may emit different sounds and sound intensities relative to the presence/absence of the various quantities (e.g., heart function, pulse function) being detected. FIG. 3C illustrates a tactile/haptic indicator 318, such as a vibration-generating element that may vibrate at various intensities relative to the presence/absence of the various quantities (e.g., heart function, pulse function) being detected. In general, the indicators should preferably be small-scale (e.g., surface-mount style) components so as to fit in with the overall thin profile of device 300. Any combination of indicators 314/316/318 may be used. For example, a device 300 may use an indicator 314 for conveying heart information to the technician and an indicator 316 for conveying pulse information to the technician. Alternatively, two indicators 314 can be used, one for indicating heart function and the other for indicating pulse function. Thus, use of a plurality of any one indicator type (314/316/318) is envisioned, or any other desired mix and match combination. The LEDs used for 314 may be single-color or multi-color. Varying the brightness intensity of the single color LED may be used as a means to convey the strength/weakness of a detected pulse. For example, dim brightness may be used for weak detections, and high brightness for strong detections. Or in the case of a multi-color LED, the strength of a detected pulse may be green for strong and red for weak. Similarly, for indicators 316 and 318, the strength of the sound/vibration may be correlated to the strength of the detected heart/pulse parameters. This then provides a dynamic manner for the technician to judge the presence/absence (e.g., strength/weakness) of the detected heart/pulse parameters. For example, as the sensors are continuously acquiring new heart/pulse data, the indicators respond in accordance, thereby effectively reflecting real-time heart/pulse detections. In one scenario, a patient may at first have a strong heart/pulse function and then suffer a failure such that the heart/pulse functions drop. The indicators can account for this due to the dynamic display/audio/haptic feedback function(s). The sudden drop in heart/pulse function may be reflected, for example, by an LED (e.g., 314) turning from green (good status) to red (bad status), providing an instant visual cue to the technician. For example, various detected strengths may be correlated to a color scale/spectrum (green=strong/high, yellow=moderate, red=weak/low) and likewise scales for the sound/haptic feedback may be keyed to the detected heart/pulse strengths. For example, piezoelectric or other electromechanical devices that make sound/vibrate may be used for 316, 318. These various feedback devices may assist the user/technician in more accurately determining presence/absence of a pulse, strength/weakness of a detected pulse, etc., in effective real-time. The indicators can be configured in any manner sufficient to relay the desired subject information to the technician/user. While FIGS. 3A, 3B and 3C are shown relative to the substrate/pad embodiment, such indicators (314, etc.) as described herein may likewise be used in the strap embodiment of FIG. 1E (e.g., the indicators may be built into housing 140).

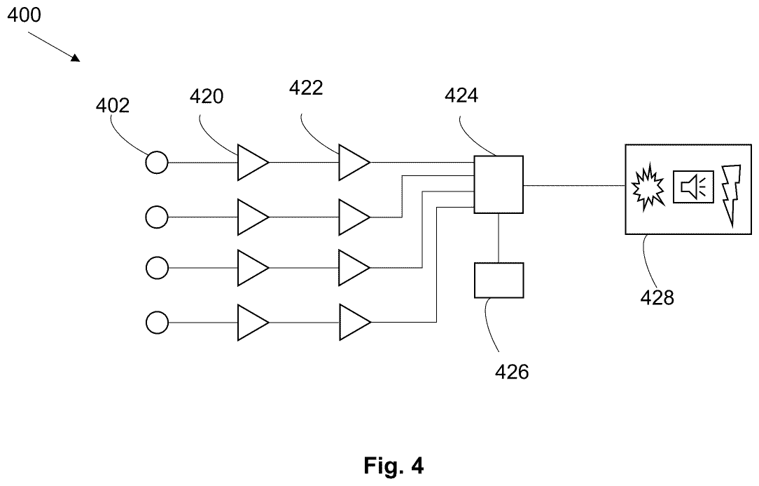

FIG. 4 illustrates a circuit schematic for a pulse detection device 400 utilizing a built-in indicator such as described for the embodiments of FIGS. 3A-C. As shown in FIG. 4, one embodiment of a circuit of pulse detection device 400 may comprise amplifying the output from each sensor 402 via an operational amplifier 420 (op-amp). The sensors may generate/output a small amplitude signal which is capable of being converted to a larger amplitude signal by way of the op-amp, for passing on to another circuit element such as a comparator 422, for example a zero-crossing detector. The comparator is capable of converting the signal(s) from the op-amp 420 for input into discrete inputs of a microcontroller 424 where their individual processed/converted sensor data is evaluated. Signal cycle measurements may be conducted and measured accurately by counting pulses of the microprocessor's clock 426 that occur during any given period. The output from the microcontroller may then be used to define the response of the indicator(s) 428 in the manner described above in connection with FIGS. 3A-C. For example, the output from the microcontroller 424 enables dynamic status indication of the sensed heart/pulse functions of the patient so that the technician has real-time indication of such patient status. The output to the indicator may be configured to trigger a response in the indicator that correlates to the determinations made as to the physiological status (e.g., pulse, no pulse) based on the sensor data.

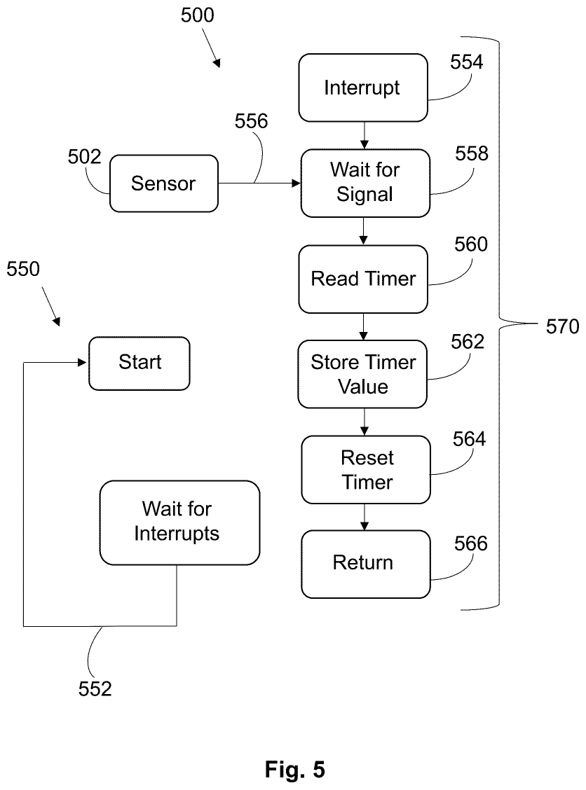

FIG. 5 illustrates a process flow for a control program executed, for example, by a microcontroller (e.g., 424 in FIG. 4) of a detection device 500. The output of sensor(s) 502 is utilized in connection with the control program. With reference to FIG. 4, the microprocessor clock may have a high and precise frequency (e.g., MHz), so that any resulting period measurement is extremely accurate. The microcontroller chip can be configured so that each of the sensor inputs generates its own interrupt schedule in the microcontroller program. These interrupts cause the microcontroller to immediately execute a special subroutine in its program where a timer/counter for each sensor can be read and reset. The action of this reading takes on the order of less than a microsecond, and program execution can then immediately resume for any other interrupts. In this way, the detections of all the sensors can be measured simultaneously with the same microcontroller. As shown in FIG. 5, a main control program 550 of the microcontroller operates in a continuous loop 552 waiting for interrupts 554 to happen. Input signals 556 from the sensors 502 to the microcontroller are set up in the software as an external interrupt that triggers the program to execute an interrupt routine 570 at any suitable time (e.g., depending on a characteristic of an input signal (e.g., 556, such as a low-to-high transition or other signal feature)). Part of the routine includes step 558 that waits for signals. Each of the input sensors 502 is connected to its own dedicated controller input line in the manner shown in FIG. 4, and has its own dedicated interrupt routine in the program so that each sensor 502 can be evaluated independently. Within routine 570 is a dedicated timer in the microcontroller, read at step 560, and its value being stored at step 562 in the memory of the microcontroller. Then the timer value is reset at step 564 to zero (although the timer continues to run, timing again from zero, and does not stop) and program execution is returned at step 566 to the main program loop 550. This subroutine 570 is executed by the microcontroller in rapid fashion (e.g., a fraction of a microsecond) and does not affect the accuracy of any time/period measurements. In this way, every sensor triggers an interrupt at every occurrence of the keying signal feature (e.g., rising edge) and the time that is stored at step 562 in the memory is always equal to the period of the corresponding sensor, or the time of one complete cycle of sensing. The frequency of a wave is f=1/T, where f is the wave frequency (e.g., in Hz) and T is the period (e.g., in seconds). It is these values (T) that can be transmitted, one for each sensor.

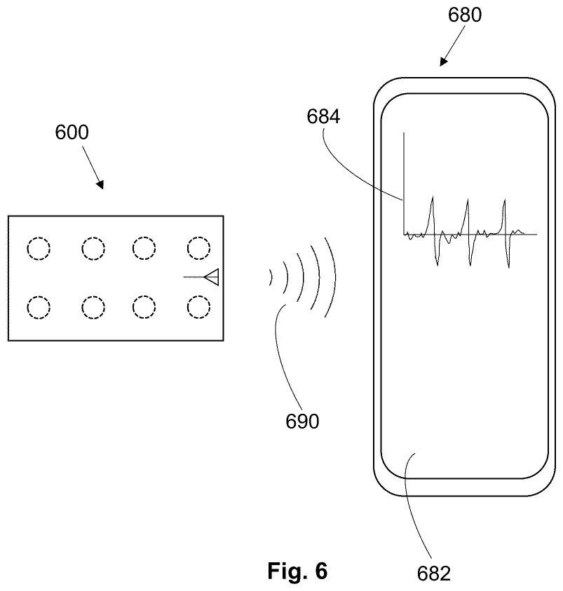

FIG. 6 illustrates an alternate embodiment where notification of the heart/pulse detections is provided to the technician via a device that is associated with but separate from the pulse detection device 600 itself—such separate device 680 being a (e.g., mobile) computer terminal. As shown in FIG. 6, pulse detection device 600 wirelessly communicates the results of the sensor detections to a remote device 680, such as a mobile phone/tablet. The remote device 680 includes the necessary hardware and software to have interoperability with the pulse detection device 600, including, for example, a display/screen 682 that is configured to display the corresponding software (e.g., application or “app”) that has a Graphical User Interface (GUI) for conveying results such as shown by GUI 684. The pulse detection device includes communication hardware and software to enable wireless communication 690 with the remote device 680 (where the remote device 680 likewise includes the necessary hardware and software to receive and process such communications). The remote device 680 may be a mobile (e.g., handheld, portable) device, or can be a medial display unit (e.g., medical monitor) capable of receiving and displaying the received data from the transmission antenna of the pulse detection device 600. The remote device 680 may comprise its own processor, memory, programs, and display, including any receiving circuitry (e.g., antenna) necessary to receive and process the transmission from the pulse detection device. The separate device 680 may be a separate monitoring device such as a smart phone, tablet, smart watch, or other smart-wear device (e.g., smart glasses, Head-Up display (HUD), ear buds, etc.). For example, in the case of the separate device 680 being a smart watch, the user/technician can see near instant (and dynamic) results of the output of the pulse detection device on their wrist in a convenient manner (e.g., no need to hold the separate device 680 in-hand). While 600 in FIG. 6 represents the substrate/pad embodiment, the strap embodiment of FIG. 1E is likewise envisioned relative to FIG. 6 (e.g., the various wireless transmission components can be in housing 140, such as on a circuit board in housing 140).

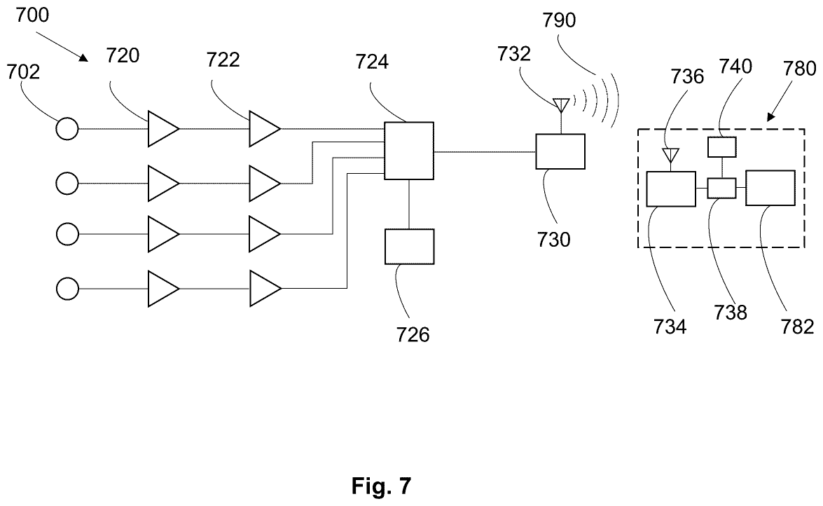

FIG. 7 illustrates a circuit schematic for a pulse detection device 700 according to the embodiment described for FIG. 6 (e.g., capable of wireless transmission to a remote device). FIG. 7 has a similar arrangement to that of FIG. 4, except also depicts various wireless aspects and aspects of the remote device. As shown in FIG. 7, one embodiment of a circuit of pulse detection device 700 may comprise amplifying the output from each sensor 702 via an operational amplifier 720 (op-amp). The sensors may generate/output a small amplitude signal which is capable of being converted to a larger amplitude signal by way of the op-amp, for passing on to another circuit element such as a comparator 722, for example a zero-crossing detector. The comparator is capable of converting the signal(s) from the op-amp 720 for input into discrete inputs of a microcontroller 724 where their individual processed/converted sensor data is evaluated. Signal cycle measurements may be conducted and measured accurately by counting pulses of the microprocessor's clock 726 that occur during any given period. The output from the microcontroller may then be output to communication (e.g., transmission) circuitry 730 of pulse detection device 700, so that data representing the sensor detection outputs is transmitted via antenna 732 and wireless communication 790 to remote device 780. The remote device 780 has communication (e.g., receive) circuitry 734 and antenna 736 capable of receiving and processing the data transmitted from pulse detection device 700. The remote device 780 includes, for example, its own processor 738, memory 740, and display 782. For example, the output from the microcontroller 724 enables dynamic status indication of the sensed heart/pulse functions of the patient so that the technician has real-time indication of such patient status. The use of an external (mobile terminal) device such as in FIGS. 6, 7 may be in addition to the indicators 314 etc. described above, or used in alternate to the indicators (e.g., no indicators need be present on/in the pad itself, since the ultimate user-perceivable results will be displayed on the terminal screen, such as via elements 682/684 in FIG. 6).

Relative to the features of the embodiment in FIGS. 6 and 7, FIG. 8 illustrates a process flow for a routine for the wireless communication of data from pulse detection device 800 to remote device 880, including a process flow for the display of a GUI on the remote device regarding graphical representations of the received data, to be perceived by the technician. Initially, the same general process flow in FIG. 5 (relative to FIG. 4) applies to the embodiment of FIG. 7, except that the ultimate output from the pulse detection device in FIG. 7 goes to the remote device, instead of being used to trigger the indicators as shown in FIG. 4. The indicators of the embodiment of FIG. 4 may however be used in combination with the wireless embodiment features of FIGS. 6 and 7. Thus it is envisioned that various aspects of these embodiments may be combined (or used separate).

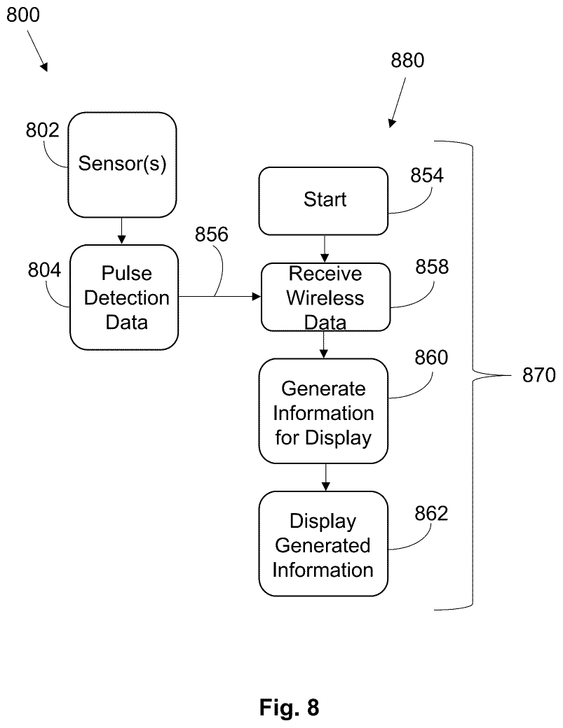

FIG. 8 shows a process flow for transmission of pulse detection data 804 from sensors 802 of pulse detection device 800, wirelessly (e.g., in the manner shown at 790 in FIG. 7) to the remote device 880, to be received and processed by the remote device via routine 870. Routine 870 shows a start of the routine at 854, where the remote device is looking for new data on a periodic basis from wireless transmission(s) 856. This for example may run as a loop to constantly look for new incoming data. The data from the pulse detection device is received at step 858 by the remote device 880 in the manner shown and described relative to FIG. 7. The received data is processed and placed into a form capable of being generated for display on the remote device at step 860. At step 862, the corresponding program of the remote device displays the generated information from the received and processed sensor data, for example to be displayed as shown at 684 in FIG. 6. The display of the data via the GUI can include any corresponding use of images or graphics, etc., so long as it is capable of conveying to the technician viewing the remote device the heart/pulse status of the patient. This includes but is not limited to graphs, or other visual/audible/tactile indications, etc., and may be updated in real-time. Text may also be used. For example, in the case of where the sensor data is transmitted to a device with a display, the display may show text such as “PULSE DETECTED”, “WARNING”, “ERROR”, “DEFIBRILLATE NOW” or other status text to inform the technician of various conditions or to prompt the technician to take action.

Another goal of the device described herein is for the device to approach the “gold standard” reliability and accuracy of (invasive) arterial blood pressure detection, but without the invasiveness. The device described herein can accomplish this by way of coding/algorithms and other programming that may utilize pulse models, where such models may be sourced from known, highly accurate data, and where such models may, over time, learn and improve from various source data, to aid in more accurate determination of a pulse from detected pressure values.

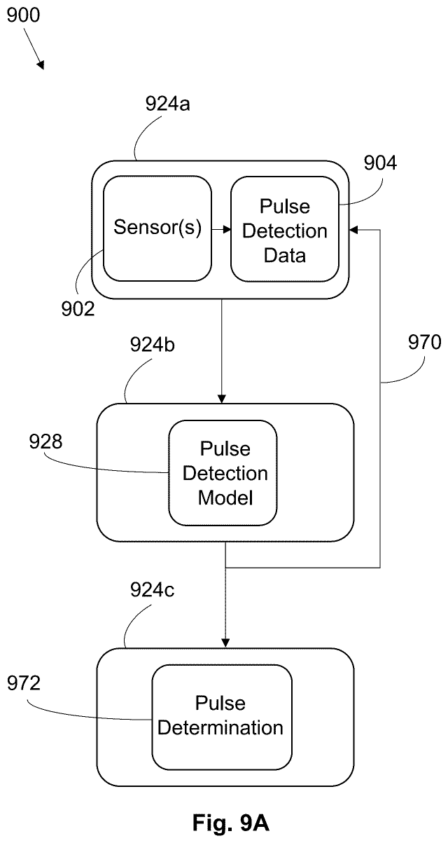

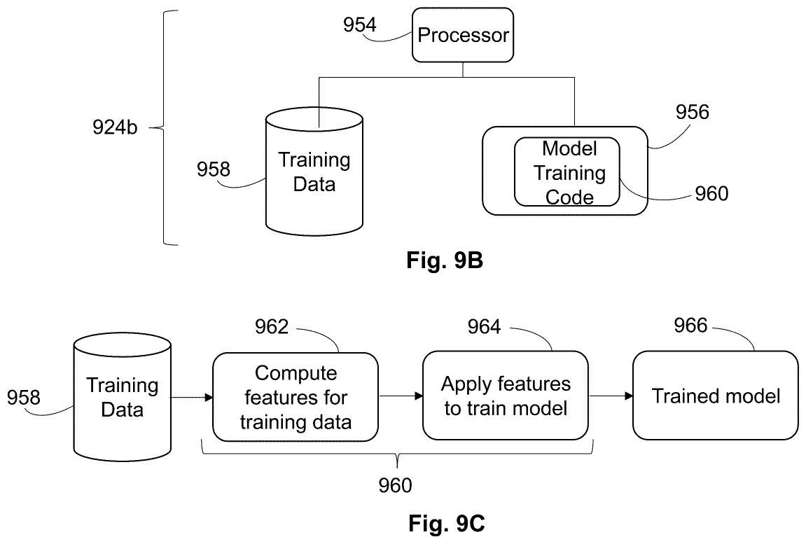

FIG. 9A illustrates another embodiment of a pulse detection device in which the ability to match the accuracy of conventional arterial techniques is a focal point. To accomplish such high accuracy, an algorithm may be used to convert detected pressure to blood pressure or vice versa, in a manner consistent with arterial techniques. Pulse detection device 900 includes a detection unit 924a comprising hardware such as sensor(s) 902 and other associated compute components, circuitry, etc. as described herein, including any software necessary for acquiring/processing, and outputting pulse detection data 904 derived from the physical pulse sensed by the sensor(s) 902 to modeling unit 924b. Modeling unit 924b may contain the necessary compute components, software/code, and a (software) model serving as a pulse detection model 928. As described in more detail herein, this model 928 may initially be trained on real-world or other baseline data (e.g., from other (e.g., invasive) blood pressure detection techniques) to establish a starting point for the training of the model, and then continuously be trained by updating the model 928 with other data, until a desired accuracy convergence is realized (e.g., the model is trained). The model 928 may be based on other/additional parameters such as the location of the device on the body, specific or generalized patient data (e.g., height, weight, age, etc.), medical conditions, and/or other physiological parameters. The model may comprise the various parameters being scaled or otherwise weighed or relationally coordinated to one another. In some embodiments, the output of model 928 may be fed back via 970 into the detection unit 924a to enable on-the-fly adjustments to the sensed data 904 based on the model, while the unaltered sensor data may still be separately fed into the model for continued updating/learning of the model. For example, newly acquired sensor data may be entered into the model 928 to update the trained model even further. With each iteration of new data, the model learns and grows stronger/more accurate, and is better able to produce more accurate results. The output from the modeling unit 924b may flow to determination unit 924c which may include its own computer components and software/code to make a pulse determination 972. The pulse determination 972 may be the ultimate determination data used to provide the user with the pulse status (e.g., no pulse, weak pulse, etc.). For example, the pulse determination 972 may, once the output from unit 924b is processed, end up being reflected as the visual display readout as shown in FIG. 6 that informs the user as to the pulse status.

FIG. 9B and FIG. 9C illustrate the training of the model 928. FIG. 9B shows the modeling unit 924b of device 900 including processor(s) 954, memory 956, and a source of training data 958. Code 960 is stored in memory 956, which can be a non-transitory computer-readable storage medium, and code 960 can take the form of processor-executable instructions that are executed by processor(s) 954 to cause the processor to train model 928. The real-world sensor data from sensors 902 can be compared to the model 928, and so that a degree of confidence as to accuracy can be realized. For example, the real-world sensor data may be compared to the model, and if the real-world data matches closely with the model, a high accuracy of the real-world data can be assigned, which means that a high accuracy as to the detected pulse is also assigned and communicated to the user by way of the display such as in FIG. 6. If the accuracy fails to meet the desired threshold relative to the model, the user may be prompted to re-located or otherwise adjust the device relative to the patient. The training data 958 may comprise data that describes a plurality of known pulse detection parameters, including those acquired/derived from arterial techniques and as described above (e.g., other patient/physiological parameters, etc). In a highly specific example, the training data may comprise arterial blood pressure data from a 56 year old male weighing 200 lbs. This data may be sourced from various databases that include such information, or from other (e.g, anonymized/randomized) medical records, etc.

FIG. 9C illustrates an example process flow to be carried out by code 960 when executed by processor 954. The known data from source data 958 is accessed, and the processor can generate a plurality of features for this training data (step 962). The source data may comprise data representing a plurality of known pulse events (e.g., cardiac arrest, etc.). Examples of features that can be derived include one or more of the following in any combination of information relating to aspects such as: blood pressure level and other physiological parameters (e.g., blood type, etc.). The generated features are applied to train model 928 (step 964) for analyzing/modifying/processing the real-world obtained data (e.g., 904) to arrive at non-invasive pulse detection accuracy using device 900 that is on par with conventional arterial accuracy. For example, once a desired feature set is applied to the model, the model may be considered trained, and thus saved (step 966), and capable of being used. The real-world data 904 can be compared or run through the model to determine if the real-world data is within a certain degree of confidence relative to the trained model. For example, when the real world data is determined by the software of the device to closely match the model (which may have been trained on highly accurate arterial data), it can be relied upon as an accurate source relative to the presence/absence and/or level of a detected pulse. Or the sensed data can be ran through the model to convert it to other pressure data (e.g., the raw sensed data can, by the techniques described herein, be correlated to the known accurate data and converted (e.g., a detected pulse pressure can be extrapolated to blood pressure, or vice versa)).

Further regarding FIGS. 9A to 9C, the pulse detection unit 924a, the modeling unit 924b and the pulse determination unit 924c may each comprise units of a common control circuit, or may be separate circuits that interface with one another (e.g., all circuits/units may be resident on the device 900 itself, or some (e.g., 924b, 924c) may be remote (e.g., on separate devices, in the cloud, etc.)). The units 924a-c may comprise all the necessary/dedicated/specific hardware/software as described herein for the particular task(s) executed by the respective units.

As described herein, the control circuit(s) can include a processor(s) that divides decision-making functionality described herein, and the processor can take the form of a field programmable gate array (FPGA) or application-specific integrated circuit (ASIC) which provides parallelized hardware logic for implementing such decision making. The FPGA and/or ASIC (or other compute resource/architecture) can be included as part of a system-on-chip (SoC). However, it should be understood that other architectures for the control circuit could be used, including software-based decision-making and/or hybrid architectures which employ both software-based and hardware-based decision-making. The processing logic implemented by the control circuit can be defined by machine-readable code that is resident on a non-transitory machine-readable storage medium such as memory within or available to the control circuit, and the code can take the form of software or firmware that define the processing operations discussed herein for the control circuit. The code can be stored/downloaded onto the control circuit in a variety of manners, including but not limited to wired/wireless connections. This can enable over-the-air updating which would be particularly useful for a device that may be used in the field.

FIGS. 9A to 9C reflect aspects for code/algorithms that can simulate, compare, and learn relative to conventional arterial blood pressure techniques, so that the device can approach a degree of detection accuracy on par with invasive arterial techniques but without the invasiveness. Thus, the code/algorithms represented in FIGS. 9A to 9C serve as a technical feature that improves upon conventional techniques in the art. The techniques disclosed herein may use various models and machine learning to steadily grow and improve the algorithm to ultimately output highly accurate pressure data mirroring that of conventional arterial techniques, and therefore represents a technical solution to the technical problem of accurate pressure detection, while avoiding conventional drawbacks such as invasiveness. The code/algorithms/(machine) learning of the present device may be realized via hardware (controllers, ICs, etc.) and software that may utilize processing techniques such as parallel processing, efficient storing of data in memory, etc. This is a practical application of computer technology applied in the context of the technical field of medical devices such as blood pressure detection devices, and represents a technical innovation in the medical field that is significantly more than the conventional systems in the art because it can produce pressure detection results on par with conventional techniques but without conventional drawbacks such as invasiveness. The data steps described herein cannot be performed in the human mind because real-time calculations and determinations are being made, and, in the context of blood pressure detection, a determination as to a patient's status needs to be made within mere seconds to be able to provide adequate treatment. For example, in an emergency situation there is not time for a human to extrapolate the vast amount of detection data being parsed by the hardware/software and processing steps disclosed herein. The techniques described relative to FIGS. 9A to 9C may be utilized in any of the other embodiments described herein.

Relative to what has been described herein, wireless transmissions may made by way of protocols such as Bluetooth, Wi-Fi, NFC or the like, alone or in combination. Many microcontroller chips include integral wireless data transmitters onboard which obviates the need for a separate chip. Thus, the wireless functionality may be by way of a separate chip or be integral within the microcontroller. The wireless data transmitter then transmits the (sensor) values via electromagnetic radio waves from an integral antenna to a distant wireless data receiver/transceiver, which could be a smartphone, a mobile tablet/laptop computer, a wearable device, or any other device with a wireless data receiver/transceiver or access to Wi-Fi. Communication may be one-way or two-way capable. The display can be the screen of an existing smartphone, smartwatch, smart glasses, computer, medical terminal/monitor, or a dedicated, wearable receiver-display unit using a graphic screen such as a liquid crystal display (LCD), organic light-emitting diode (OLED), or any other form of electronic graphic display.

The pressure sensor(s) may be based, for example, on absolute, gauge, or differential measurement modes. The sensing of the sensors may be by way of resistive, capacitive, piezoelectric, optical, or microelectromechanical system (MEMS) sensing. The sensors may include but are not limited to strain gauge-type sensors, solid-state sensors, and micromachined silicon (MMS) sensors. The (pressure) sensors may include a differential pressure transducer, a piezoelectric pressure sensor (e.g., fully piezo-resistive silicon pressure sensor), a MEMS pressure sensor, a diaphragm-based pressure sensor (e.g., membrane-based pressure sensor), etc. For example, thin- or thick-film sensor technologies may be used (e.g., metal thin-film, ceramic thick-film). Variable capacitance pressure sensors may be used, and other resistive or capacitive properties may be utilized to aid in pressure sensing. The sensors may comprise technology comprising piston technology, mechanical deflection, piezoelectric materials, vibrating elements, be semiconductor-based, and the like. Also, sensor materials comprising various (e.g., semiconducting) materials capable of use in pressure-sensing applications may be utilized (e.g., organic or polymer semiconductors). Materials capable of being used in flexible sensors, such as graphene-based materials (e.g., graphene oxide), ZnO, and carbon-based materials (e.g., carbon black (CB), carbon nanotubes) may be used. In a preferred embodiment the pressure sensors may comprise surface-mount ICs amenable for use in wearable electronics.

Further regarding a diaphragm-based pressure sensor, and with reference to FIGS. 1C and 1D, the sticking of the pad to the patient via the adhesive surface of the pad may provide sufficient pressing force for the membrane to accurately sense a pulse. However, it is also envisioned that a technician may apply additional external force (e.g., by pressing their hand/finger) on the pad to achieve the desired pressing force for pulse detection via the membrane. For example, and without limitation, the diameter of the head of the embodiment in FIGS. 1C and 1D may be similar to the diameter of a US quarter coin. Larger head diameters (e.g., ˜2 inches in diameter or more, but still sized to be completely contained within a perimeter/circumference of the pad) may also be used, depending, for example, on the sensing or sizing parameters desired. The head may comprise a certain volume for desired pressurization. The head may comprise a material of a certain weight to achieve the desired contact with a patient's body (e.g., skin). To this end, relative to the sensor array shown in FIGS. 1A, 2, etc., the desired amount of membrane-type sensors can be configured as part of the pad. For example, only one membrane sensor such as in FIGS. 1C, 1D may be used, with all pressure detection being by way of the single sensor. If multiple (pressure sensors) used, the sensor outputs may analyzed relative to one another to arrive at a unitary determination as to what the sensor data suggests is the physiological state. For example, multiple sensors outputs may be averaged, or otherwise compared relative to one another to arrive at a strength of confidence in the presence/absence of a pulse. For example, for a pad comprising an array of three (pressure) sensors, the outputs of the sensors may be analytically compared to arrive at the ultimate determination of whether or not the sensors detected pressure representative of a pulse. Because the sensors may return different results depending on their placement relative to a vein (or other portion) of the patient, one sensor may return stronger results than another (e.g., adjacent) sensor. Software programs dedicated to analyzing the sensor data may therefore be implemented and used so as to arrive at the most accurate overall result based on the multiple sensor outputs. In other words, the plurality of sensors outputs may be harmonized, after processing/comparison, to be indicative of one result, e.g., presence, or absence, of a pulse. Machine learning may be used in this regard to learn, over time, which types/levels of pressures detected by the sensors are most indicative of the various physiological pulse possibilities, and to make accurate determinations from a plurality of sensor data.

The LEDs and photosensors may be of any type well-suited for photoplethysmography applications, such as, but not limited to, green LEDs. The light components should be well-suited for emitting and detecting light that is best-suited for detection of heart/blood flow-related physical parameters of living things (human or otherwise). For example, the photodetectors may be tuned or formed to be a best match for the wavelength of light of the preferred LEDs. While LEDs and corresponding photodetectors are preferred, other optical implements/components for detection of heart rate and/or blood flow may be used, such as, but not limited to, IR light sources, and/or light sources of other wavelengths, or that use other optical properties/phenomena.

The techniques disclosed herein are for determining the presence of a (e.g., cardiac) pulse in a patient by evaluating physiological signals in the patient. In certain embodiments described herein, a detection device is constructed to include a sensor system comprised of one or more sensors, for example arranged as an array on a substrate such as a pad or other flexible membrane. The sensor system or any portion of it can be wearable by the patient or can be attachable to the patient in any other suitable manner such as adhesive. The sensor pad, may, for example, include an adhesive backing. The pad may be a one-time use item or re-usable. The sensor system is adapted to sense various physiological signals in a patient. The physiological signals are converted into digital physiological signal data that is processed by processing circuitry in or associated with the device(s). The processing circuitry is configured to evaluate the data from each physiological signal for a feature indicative of the presence of a cardiac pulse. Using these features, the detection device determines, for example, whether a cardiac pulse is present in the patient. The detection device further includes the ability to dynamically display, in a user-perceivable manner, whether a cardiac pulse is sensed and thus considered as being present in the patient. Examples of usable physiological signals include phonocardiogram signals, electrocardiogram signals, and patient impedance signals. Also, as noted herein, embodiments of the invention may use signals obtained from piezoelectric sensors and/or other sensing devices (e.g., accelerometers) placed on the patient's body.

The physiological signal data is analyzed and evaluated to determine whether a pulse is present in the patient. This may include analyzing sensor data for features indicative of the presence/absence of a cardiac pulse and other related physiological parameters to determine whether a cardiac pulse is present based on the feature.

In a further embodiment of the invention, the detection device may include additional sensors such as electrodes to be attached to the patient. While the device is referred to herein as a pulse detection device, it is capable of detecting other parameters and therefore is not limited only to pulse detection. While the controller is in one embodiment envisioned as being part of the detection device, the controller can be offloaded or present in a device other than detection device. This may reduce complexity or cost of the detection device. For example, the detection device may instead include NFC technology or the like to transfer (sensor) data stored in a memory of the detection device to a nearby device, so that the nearby device may handle the more intense data processing aspects. Other aspects may be offloaded from the main detection device as well. For example, instead of the detection device having built-in indicators, a remote indicator station that may take the form of a display terminal such as a traffic light (red, yellow, green lights) may be in communication with the detection device and configured to receive data from the sensors of the detection device and activate the color of light corresponding to a condition (e.g., red=no pulse detected). This may provide for an embodiment that provides extremely easy visible detection of the patient status by a technician, and may be useful in loud conditions or other environments such as the back of an ambulance. For example, further regarding a minimalistic embodiment of the pulse detection device/pad, and to maximize aspects pertaining to instantaneous (or one-time) use of the pad (e.g., in an emergency situation by EMS technician), the pad may be configured to contain the bare minimum amount of components needed to acquire accurate pulse detection. For example, the pad may only have the sensors, minimum necessary signal transmission components (e.g., wires), and minimum IC(s) necessary for receiving the sensor data and then (e.g., wirelessly) transmitting the sensor data to a nearby receiving/remote terminal (as discussed herein) that performs processing/analysis of the raw sensor data and displays the results thereof accordingly. By minimizing the amount and complexity of components in the pad itself, this reduces the pad to its most simplistic functional (and structural) configuration, lending to improvements in aspects such as pad adhesion, pad flexibility, pad weight, one-time usage, and the like. For example, circuitry 108 could be a minimalistic as possible, providing only for transmission of the raw sensor data to another circuit and/or IC that simply offloads the raw data to an external device for processing. With reference to FIGS. 4 and 7, the microcontrollers 424/724 may comprise one or more microcontrollers, with one microcontroller being located in the pad itself and used merely for basic compiling/transmission of the sensor data, and another microcontroller being located in a separate device for processing of the sensor data. Thus, FIGS. 4 and 7 are not limited to a scenario where all of the components (e.g., 420/720, 422/722, 424/724, etc.) are located in the pad device itself. The components may be allocated as desired across multiple associated devices. Thus, for the above-discussed minimalistic embodiment of the detection pad, as few components as possible (e.g., as few as possible of those shown in FIGS. 4 and/or 7) may actually be located in the pad device itself. On the other hand, in a scenario where the pad is meant for more long-term use (such as in a continuous detection, at-home use (or a prolonged hospital stay)), the pad may have a more complicated design, where more components are present within and/or in association with the pad. Thus, the complexity of the pad design may be varied based on its intended usage.

In the present disclosure, all or part of the units or devices of any system and/or apparatus, and/or all or part of functional blocks in any block diagrams and flow charts may be executed by one or more electronic circuitries including a semiconductor device, a semiconductor integrated circuit (IC) (e.g., such as a processor, CPU, etc.), or a large-scale integration (LSI). The LSI or IC may be integrated into one chip and may be constituted through combination of two or more chips. For example, the functional blocks other than a storage element may be integrated into one chip. The integrated circuitry that is called LSI or IC in the present disclosure is also called differently depending on the degree of integrations, and may be called a system LSI, VLSI (very large-scale integration), or VLSI (ultra large-scale integration). For an identical purpose, it is possible to use an FPGA (field programmable gate array) that is programmed after manufacture of the LSI, or a reconfigurable logic device that allows for reconfiguration of connections inside the LSI or setup of circuitry blocks inside the LSI. Any database/recording medium/storage medium or the like referenced herein can be embodied as one or more of ROMs, RAMs, optical disks, hard disk drives, other solid-state memory, servers, cloud storage, used in isolation or in combination, and so on and so forth. Furthermore, part or all of the functions or operations of units, devices or parts or all of devices can be executed by software processing (e.g., coding, algorithms, etc.). The software is recorded in a non-transitory computer-readable recording medium, such as one or more ROMs, RAMs, optical disks, hard disk drives, solid-state memory, servers, cloud storage, and so on and so forth, having stored thereon executable instructions which can be executed to carry out the desired processing functions and/or circuit operations. For example, when the software is executed by a processor, the software causes the processor and/or a peripheral device to execute a specific function within the software. The system/method/device of the present disclosure may include (i) one or more non-transitory computer-readable recording mediums that store the software, (ii) one or more processors (e.g., for executing the software or for providing other functionality), and (iii) a necessary hardware device (e.g., a hardware interface).

The embodiments were chosen and described in order to best explain the principles of the disclosure and its practical application to thereby enable others skilled in the art to best utilize the disclosure in various embodiments and with various modifications as are suited to the particular use contemplated. Aspects of the disclosed embodiments may be mixed to arrive at further embodiments within the scope of the invention.

As various modifications could be made in the constructions and methods herein described and illustrated without departing from the scope of the disclosure, it is intended that all matter contained in the foregoing description or shown in the accompanying drawings and/or appendices shall be interpreted as illustrative rather than limiting. Thus, the breadth and scope of the present disclosure should not be limited by any of the above-described exemplary embodiments, but should be defined only in accordance with the following claims appended hereto and their equivalents.

What is claimed is:

1. A detection apparatus comprising:
a substrate having a top surface and an opposite bottom surface, the bottom surface of the substrate being adapted and configured to be placed on a subject's skin;
a sensor integral with the substrate, the sensor comprising one of a optical sensor and a pressure sensor, the sensor being configured to sense a parameter of the subject to which the substrate is placed; and
circuitry integral with the substrate, the circuitry including a processor, the processor being in operative communication with the sensor, the processor being configured to receive and process the output of the sensor and to output data representing the at least one parameter of the subject to which the substrate is placed, such that the status of the subject can be indicated to a user of the detection apparatus.

2. The detection apparatus of claim 1, wherein the parameter of the subject is a pulse parameter of the subject.

3. The detection apparatus of claim 1 wherein the status of the subject is a physiological status of the subject.

4. The detection apparatus of claim 1 wherein the sensor is adapted and configured to extend outward from the bottom surface of the substrate when the substrate is applied to the subject.

5. The detection apparatus of claim 1, further comprising a remote terminal adapted and configured to interface with the processor, the remote terminal being configured to receive the transmitted data representing the status of the subject, process the received data representing the status of the subject, and indicate a user-perceivable representation of the processed data.

6. The detection apparatus of claim 5 wherein:
the remote terminal comprises a processor, a receiver; and an indicator,
via the receiver, the remote terminal receives the transmitted data representing the status of the subject
via the processor of the remote terminal, the remote terminal processes the received data representing the status of the subject; and
via the indicator, the remote terminal indicates a user-perceivable representation of the processed data, such that the indicator indicates the status of the subject to a user of the detection system.

7. The detection apparatus of claim 1, wherein the sensor is one in a plurality of sensors.

8. The detection apparatus of claim 1, wherein the sensor comprises one of an LED and photosensor pair.

9. The detection apparatus of claim 1, wherein the circuitry is adapted and configured to receive, store, and/or analyze data acquired by the sensor for determination of (i) the parameter and/or (ii) a status of the subject.

10. A method for detecting a parameter of a subject, the method comprising:
attaching a detector to the subject by applying a bottom surface of a substrate of the detector to the subject's skin;
acquiring a parameter of the subject via a sensor of the detector; and
indicating the status of the subject based upon the parameter.

11. The method of claim 10, wherein the step of acquiring the parameter of the subject via the sensor of the detector includes:
receiving and processing an output of the sensor with a processor associated with circuitry integral with the substrate.

12. The method of claim 11, wherein the step of indicating the status of the subject based upon the parameter includes:
with a processor associated with circuitry integral with the substrate, outputting data representing the parameter of the subject to which the substrate is placed such that the status of the subject can be indicated to a user of the detection apparatus.

13. The method of claim 12, wherein the step of indicating the status of the subject based upon the parameter includes indicating a physiological status of the subject.

14. The method of claim 12, wherein the step of indicating the status of the subject based upon the parameter includes indicating a pulse parameter of the subject.

15. The method of claim 10, further comprising:
providing a remote terminal with a processor, a receiver; and an indicator,
via the receiver, receiving transmitted data representing the status of the subject;
via the processor of the remote terminal, processing received data representing the status of the subject; and
via the indicator, indicating a user-perceivable representation of the processed data, such that the indicator indicates the status of the subject to a user of the detection system.

16. The method of claim 10 wherein the step of attaching the detector to the subject by applying the bottom surface of the substrate of the detector to the subject's skin includes configuring the substrate in a manner such that the sensor extends outward from the bottom surface of the substrate when the substrate is applied to the subject.

17. The method of claim 10, wherein the step of acquiring the parameter of the subject via the sensor of the detector includes configuring an LED to provide output representative of the status of the subject.

18. The method of claim 10, wherein the step of acquiring the parameter of the subject via the sensor of the detector includes configuring an pressure sensor to provide output representative of the status of the subject.

19. The method of claim 10, wherein the step of acquiring the parameter of the subject via the sensor of the detector includes configuring a photosensor pair to provide output representative of the status of the subject.

20. The method of claim 10, wherein the step of acquiring the parameter of the subject via the sensor of the detector includes configuring a plurality of sensors to provide output representative of the status of the subject.
